(19) United States
(12) Patent Application Publication
(10) Pub. No.: US 2005/0239078 A1
(43) Pub. Date: Oct. 27, 2005

(54) Sequence tag microarray and method for detection of multiple proteins through DNA methods

(75) Inventor: N. Leigh Anderson, Washington, DC (US)

Correspondence Address: LARGE SCALE BIOLOGY CORPORATION, 3333 VACA VALLEY PARKWAY, SUITE 1000, VACAVILLE, CA, 95688, US

(73) Assignee: LARGE SCALE PROTEOMICS CORPORATION, a Division of Large Scale Biology Corporation, Vacaville, CA (US)

(21) Appl. No.: 10/689,892

(22) Filed: Oct. 20, 2003

Related U.S. Application Data
(63) Continuation of application No. 09/714,671, filed on Nov. 17, 2000, now abandoned.
(60) Provisional application No. 60/166,266, filed on Nov. 18, 1999.

(51) Int. Cl. C12Q 1/68; C12M 1/34

(52) U.S. Cl. 435/6; 435/287.2

(57) ABSTRACT

Methods and reagents for simultaneously measuring the concentration of numerous proteins in a sample are described. The technique uses many antibody display phages that contain corresponding specific nucleic acid sequence tags. Binding between various proteins and antibodies is determined by simultaneous detection of the specific sequence tags using a microarray. This method is applicable even when the concentrations of different proteins differ by orders of magnitude as the nucleic acid sequence tags may be amplified.

### FIELD OF THE INVENTION

The present invention relates to the simultaneous quantification of a large number of proteins of widely differing concentration.

### BACKGROUND OF THE INVENTION

The simultaneous quantitative detection of multiple target DNA and RNA sequences has been accomplished by a number of techniques. Microarrays and blots are convenient tools for accomplishing this goal as each unique sequence has a complementary unique sequence to which it will specifically hybridize. By placing complementary nucleic acids, or the target nucleic acids, at separate and identifiable locations on the microarray or blot, the presence of nucleic acid binding is indicative of the presence of target nucleic acid present. Representative patents and publications for this technology include U.S. Pat. No. 5,143,854, Fodor et al, Science 251: 767-773 (1991), U.S. Pat. No. 5,424,186, U.S. Pat. No. 5,807,522, U.S. Pat. No. 5,569,588 and Southern, Journal of Molecular Biology 98:503 (1975).

Alternatively, the polymerase chain reaction (PCR) has been used to detect target nucleic acids wherein a particular set of primers is used to amplify a particular target. Careful control makes the process quantitative or at least semi-quantitative.

This ability to detect large numbers of nucleic acids is primarily attributable to three properties: 1) specific probes for a variety of DNA's can easily be made in any quantity with great uniformity in the form of complementary DNA sequences, 2) these probes can be arrayed spatially such that each can capture its respective binding partner target from a sample and hold it in a spatially distinct location for subsequent detection, and 3) target nucleic acids bound to the probes can be detected easily by virtue of fluorescent or other labels incorporated into the target as part of sample preparation or after binding with a labeled probe.

However, for proteins, no comparable system for simultaneous screening exists. Specific binding partners to many unknown proteins can be prepared but are not easily produced in large numbers reproducibly. For example, one can prepare an antibody to a protein and use it as a binding partner but each antibody will be prepared and/or titrated separately. Antisera inherently produce higher antibody titers for immunodominant proteins and undetectable quantities of antibody to other proteins. Typically, there is little if any correlation between immunodominance and concentration in the immunogen. Hybridoma technology may theoretically permit one to generate a monoclonal antibody against all proteins; but this process is involves laborious screening of hybridomas and titration of antibodies to obtain a usable reagent.

Numerous immunoassays are known but each detects only one or a few proteins simultaneously and thus are not suitable for large numbers of proteins. Additionally, mixtures of proteins may be in widely differing concentrations and an assay optimized for one concentration of protein is generally not optimal for another protein, which is in thousand fold greater or lesser concentration. Thus, problems remain such as to how to determining the global concentrations of all proteins in a biological sample. Western blots and similar techniques exist for detecting numerous proteins simultaneously such as antigens with mixed antibody antisera. For example, Sharma et al, Journal of Immunology 131(2) 977-83 (1983). However, such techniques do not detect low concentrations of proteins and antisera have variable titers of different antibody species. Mass synthesis of arrays of peptides are known, for example U.S. Pat. No. 5,338,665 and U.S. Pat. No. 5,498,530, but such is useful for screening only one or a few suitable binding partners capable of binding to one of the peptides present. Screening of large numbers of unknown proteins is not possible using such an array of peptides because most proteins will not specifically bind to any possible short peptide. Libraries of small molecules are also known, U.S. Pat. No. 5,338,665, and may be used as ligands. However, again, specific binding partners would need to be individually made and individual assays developed.

Various chromatographic and electrophoresis methods can fractionate protein mixtures and two dimensional gel electrophoresis is capable of simultaneously separating thousands proteins. However, such techniques are labor intensive and time consuming. While these may be useful for detecting and quantifying common proteins based on peak size and retention time or location and intensity of a spot or band, such techniques do not easily quantify rare or very low concentrations of certain proteins.

Unlike nucleic acids that may be amplified by PCR, ligase chain reaction (LCR), rolling circle amplification (RCA), strand displacement assay (SDA), NASBA and other techniques, proteins are not amplifiable. Thus, low concentrations of important proteins will be missed in a mixture of proteins. Additionally, high concentrations of other proteins interfere with an assay for a low concentration protein in the mixture.

Bacteriophages have been genetically engineered to express numerous peptide sequences on their coat protein that may be use for immunological detection. See Kang et al, Proc. Natl. Acad. Sci. 88:4363-4366 and McCafferty et al, Nature 348:552-554. The peptides may be under the control of the LSC 1 gene and with C terminus peptides (Cull et al (1992)). Antibody phage display libraries are known where different phages express a different antibody on their surface. A good review article is Winter et al, Annual Reviews in Immunology, 12: 433-455. Such antibody display phage are effective for diagnostic purposes, Millens et al, Leukemia 12(8):1295-301 (1998), preserve the idiotype of a monoclonal antibody, Houbach et al, Journal of Immunological Methods, 218:53-61 (1998) and are neutralizing to a virus, Bjorling et al, Journal of General Virology 80:1987-1993 (not prior art). While these are effective for producing affinity reagents as an alternative to antisera and hybridoma technology, such have the same shortcomings as these conventional antibodies when used in conventional immunoassay formats.

Phage display of peptide ligands has been coupled with DNA-based selection techniques for enhanced screening. Bartoli et al, Nature Biotechnology 16(11):1068-1073 (1998).

Presently, no rapid method for simultaneously and quantitatively detecting large numbers of different proteins in a mixture exists where certain proteins occur in trace amounts relative to other proteins.

### SUMMARY OF THE INVENTION

The object of the present invention is to simultaneously and quantitatively measure a large number of proteins, including low concentration proteins, in a mixture of high concentration proteins.

It is another object of the present invention to employ well-developed DNA methods for the detection of proteins by using a detection reagent containing a receptor associated with a nucleic acid sequence.

The present invention accomplishes this goal by using a mixture of a large number of unique receptors associated with a corresponding large number of unique nucleotide sequences such that each unique receptor is associated with its unique nucleotide sequence. This arrangement permits binding of ligands to be detected with the receptors followed by conventional methods for detection and quantification of a large number of unique nucleotide sequences. After the receptor is bound by a ligand analyte, the unbound receptors are separated from the bound receptors. The nucleotide sequences are then optionally separately and/or optionally amplified and quantified by conventional nucleotide detection systems such as by hybridization to arrays of complementary oligonucleotides. The quantitative measurement of unique nucleotide sequences from the bound receptors thus corresponds to the amount of target ligand in the sample.

The present invention utilizes an antibody phage display library where each different phage contains a different sequence tag unique to exactly one antibody. This reagent arrangement links unique receptors to unique nucleotide sequences. A mixture of proteins from a sample is optionally bound to a solid support and then contacted with this reagent and allowed to bind therewith. The amount of each phage binding corresponds to the amount of each protein present. The unbound antibody display phage is separated and discarded. The nucleotide sequences are then recovered and hybridized to a conventional microarray where the amount of hybridization is determined quantitatively.

The present invention also relates to amplification of at least part of the nucleic acid sequence before detection by hybridization. Since proteins are not “amplifiable”, amplification of the nucleic acid containing the sequence tag serves as a proxy for amplifying proteins, thereby permitting detection of relatively low concentration proteins. PCR and other conventional nucleic acid amplification techniques may be used. Prior to the present invention, a peptide or antibody array would not be functional for detecting proteins that are in such low concentrations and cannot be amplified to easily detectable concentrations.

### BRIEF DESCRIPTION OF THE DRAWINGS

FIG. 1 is a representation of two antibody display phage, one presenting an antibody domain (a) binding to protein target A and the other presenting an antibody domain (b) binding to protein target B.

FIG. 2 depicts how a mixture of protein targets A and B in solution adsorbs to a solid support.

FIG. 3 depicts antibody display phages binding to the adsorbed protein targets.

FIG. 4 depicts the nucleic acids recovered from the bound antibody display phages and the results of a post treatment with a restriction endonuclease to release sequence tags.

FIG. 5 depicts a microarray with the sequence tags hybridized to corresponding cells.

FIG. 6 is a schematic for generating a differential concentration determination between two samples.

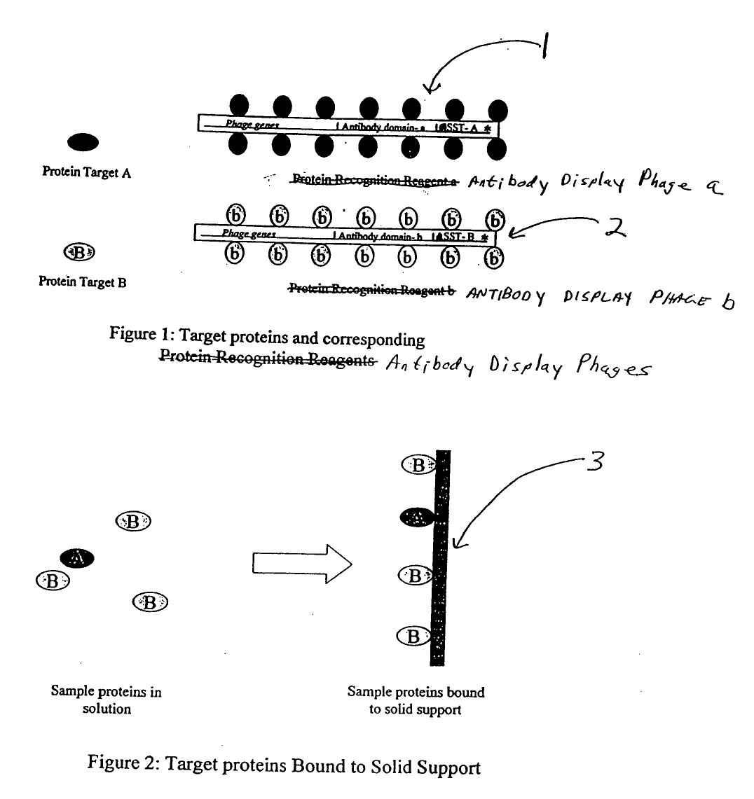

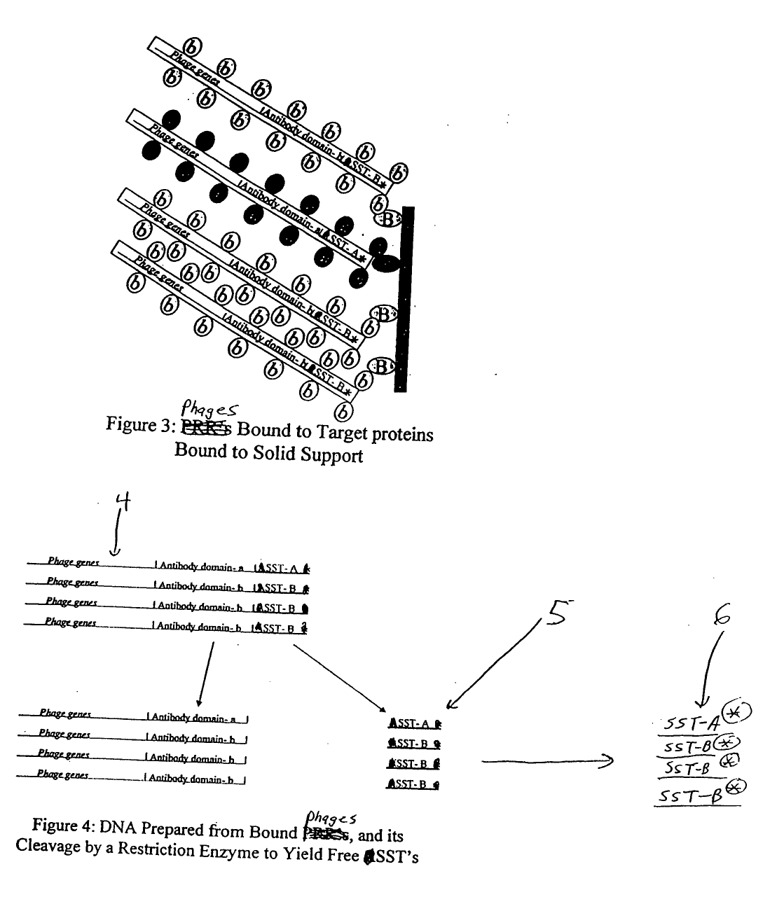

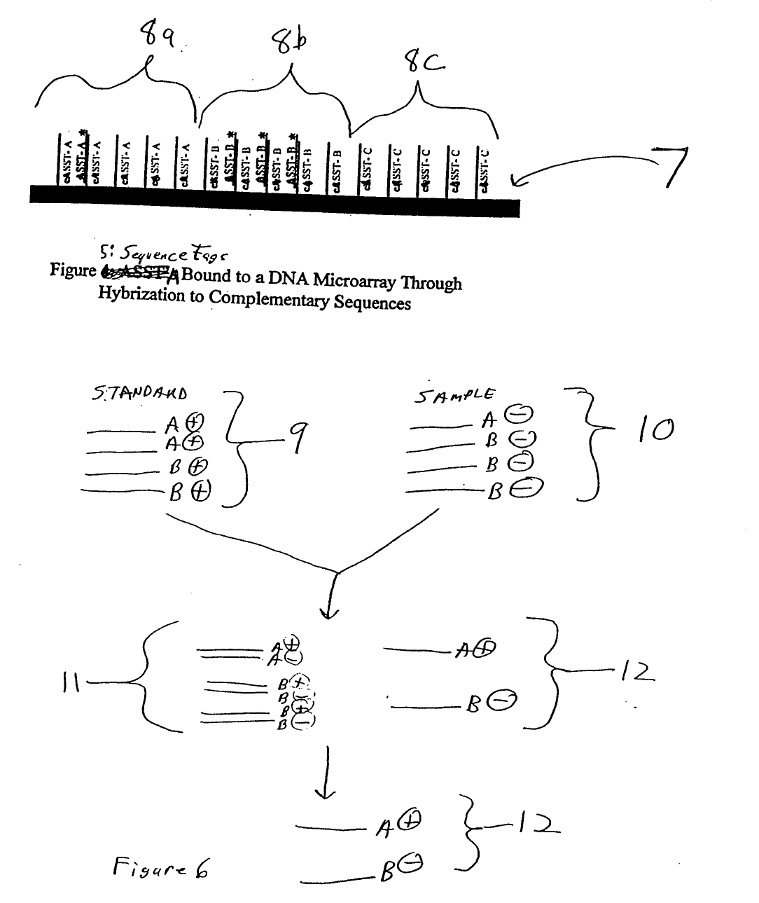

### DESCRIPTION OF THE PREFERRED EMBODIMENTS

The term “ligands” refers to chemical components in a sample that will specifically bind to receptors. A ligand is typically a protein or peptide but may include small molecules, such as those acting as a hapten. For example, when detecting a large number of proteins in a sample, the proteins are ligands.

The term “receptors” refers to chemical components in a reagent which an affinity for and are capable of specifically binding to ligands. A receptor is typically a protein or peptide but may include small molecules. For example, when using an antibody display phage library, each phage with an antibody molecule acts as a receptor.

The term “bound to” or “associated with” refers to a tight coupling of the two components mentioned. The nature of the binding may be chemical coupling through a linker moiety, physical binding or packaging such as nucleic acids are packaged inside a viral protein coat. Likewise, all of the components of a cell are “associated with” or “bound to” the cell.

An “antibody” includes antibody fragments, bifunctional, humanized, recombinant, single chain or derivatized antibody molecules. A receptor is generally not a nucleic acid.

The term “protein” is intended to encompass derivatized molecules such as glycoproteins and lipoproteins as well as lower molecular weight polypeptides.

“Small molecules” are low molecular weight organic molecules that are recognizable by the ligands or receptors. Typically, small molecules are specific binding compounds for proteins. Primers, probes and other target nucleic acid sequences may also be considered “small molecules” regardless of their size and its binding partner may have a complementary sequence.

“Labels” include a large number of directly or indirectly detectable substances bound to another compound, and are known per se in the immunoassay and hybridization assay fields. Examples include radioactive, fluorescent, enzyme, chemiluminescent, hapten, chelator, etc. Labels include indirect labels, which are detectable in the presence of another added reagent, such as a biotin label and added avidin or streptavidin, which may be labeled or subsequently labeled with labeled biotin at any point, even after hybridized to the array.

“Sequence tag” is a short sequence (typically about 13 to about 50 nucleotides) which occurs rarely if at all naturally and can serve as a unique identifier. A sequence tag may be part of an existing sequence (such as the unique sequence encoding a hypervariable region or specific binding site of an antibody) or an artificial sequence that is ligated to another nucleic acid. Artificial sequences may be inserted into the gene for an antibody molecule per se such as in a “constant” region of the antibody gene. The selection of the nucleotide sequence for the sequence tag is based upon a complementary sequence being present on the microarray for easy detection.

A “microarray” is a solid phase containing a plurality of different nucleic acids immobilized thereto at predetermined locations. The microarray generally has at least about 10, more preferably at least about 100 and even more preferably at least about 1000 different nucleic acids. By hybridizing a nucleic acid of unknown sequence to the microarray, one can determine at least part of its sequence based on its location on the microarray. While not a single solid phase, a series of many different solid phases each with a unique nucleic acid immobilized thereon is considered a microarray for the purposes of this invention. Each solid phase has unique detectable differences allowing one to determine the nucleic acid immobilized thereon.

“Hybridization” is intended to encompass specific hybridization between two single stranded nucleic acids where complete complementarity extends over a region of the two nucleic acids. One strand may be substantially longer than the other or have other moieties attached thereto provided that a sequence of complete complementarily exists which is stable under hybridizing conditions and which is unstable when that region is not completely complementary.

“Phage” refers to a large number of different viruses that are capable of being genetically modified to display a receptor or ligand specific binding moiety on their coat proteins. While bacteriophage are typically used, other viruses such as adenoviruses may be used (Douglas et al, Nature Biotechnology 17(5):470-475 (1999)).

In a preferred embodiment of the present invention, one wishes to detect the presence of and possibly the concentration of hundreds or thousands of different proteins in a biological sample. The figures exemplify the simplest example detecting two different proteins. Random sequence tags are generated by random synthesis and ligated to phage DNA. The sequence tags may be chosen to hybridize to a predetermined microarray or a microarray may be synthesized to correspond to predetermined sequence tags. An antibody display phage library is constructed by conventional means using this DNA with sequence tags (SST-A) and (SST-B). Each resulting antibody display phage (1) and (2) of the library has a unique sequence tag and a unique antibody domain incorporated into the genome of each phage with a corresponding unique antibody molecule on its surface. Each phage contains its antibody domain (a) and (b) on its surface.

The sample protein mixture (A) and (B) is incubated with a solid support (3) and is adsorbed or otherwise attached to it. An internal control may be used by adding a known quantity of a protein (either one of A or B or a new protein C). Ligands are immobilized on a solid support activated in such a way as to bind any desired ligand with high affinity. A blocking solution of a conventional unrelated protein such as gelatin, albumin or casein is added and incubated to block any additional adsorption sites on the solid support. For example, a fish skin gelatin blocking agent will block any further protein binding, primarily by covering any open solid support surfaces. A reagent containing the antibody display phage library is then added to the solid support and allowed to incubate under suitable conditions to permit the displayed antibodies (a) and (b) to bind to the immobilized proteins (A) and (B). Unbound phages are washed free thereby separating bound and unbound phage. Note that the phage quantitatively bind in accordance with the concentration of protein adhered to the solid support.

At this point, the proteins and antibodies have served their purpose of indirectly immobilizing sequence tags (SST-A) and (SST-B) and the solid support bound phage are contacted with a protease, solvent or other solution to free the nucleic acids into a liquid solution of nucleic acids (4). The nucleic acids may be cleaved to generate a pool of fragments (5) with the sequence tags and optionally labeled by any of a number of known techniques. When low concentrations are suspected, the concentration of sequence tags may be amplified by quantitative PCR or other quantitative amplification techniques.

The pool of labeled fragments containing sequence tags (6) is then hybridized to a conventional nucleic acid microarray (7). The microarray is scanned for the label (*) and the cells (8a, 8b and 8c) of the microarray with detectable label correspond to the original proteins in the sample: Likewise, the intensity of the label detected corresponds to the amount of label, hence the amount of sequence tag, hence the amount of phage, hence the concentration of original proteins in the sample.

A pool of fragments from a standard sample (9) having the sequence tags is labeled with a first label (+). A pool of fragments from a test sample (10) having the sequence tags is labeled with a second label (−). The sequence tags in one pool are designed to be complementary to the sequence tags in the second pool with respect to the same receptor. The two pools are mixed and incubated under hybridizing conditions to yield a mixture of double stranded nucleic acids (11) and single stranded nucleic acids (12). The double stranded nucleic acids are separated or inactivated to from a pool of single stranded nucleic acids (12), which represent differentially present proteins in the original sample. By contacting this with a microarray and scanning for both labels, the differential increase or decrease between samples is determined.

Within this procedure, numerous modifications and variations may be employed. The sample may be from a natural source or an artificially generated mixture of substances to be detected. Anything that will be specifically recognizable by an antibody display phage library may be detected using the present invention. For example, proteins or peptides in a biological fluid or extract may be simultaneously tested for the presence of disease markers. Alternatively, the amount of each desired organic molecule in a mixture may be simultaneously determined such as the levels of many nutrients in a food or metabolites in a cellular sample. Alternatively, past exposure to pathogens may be determined by measuring the presence and levels of antibodies in serum by generating an antibody display phage library to the idiotype of sample antibodies or a peptide display phage library.

Unlike previous analytical techniques, one does not need to first separate the ligands before quantitative and qualitative determination of a very large number of ligands simultaneously.

To allow for later separation of receptor bound ligands from unbound receptors, the ligands may be immobilized on a solid support. The solid support may be in the form of the inside of a container, a membrane, a movable strip or object within a container or preferably small beads. Commercially available magnetic, supermagnetic, paramagnetic or ferromagnetic beads are preferable as they are available in pretreated form to bind to the ligands. By the application of magnetic energy or a magnetically attractive material, the bound materials are easily recovered and in low volumes for easy concentration. By washing the solid support to remove unbound receptors while leaving receptors bound to immobilized ligands attached to the solid support effects separation.

To enhance adsorption to the solid support, the solid support may be coated with non-specific protein or peptide adsorbing material. Silica, hydrophobic moieties and C18 derivatized solid supports are known in the field of column chromatography to adsorb proteins. The same may be used as the solid support or a coating on the solid support for the present invention. Elution may be accomplished using an organic solvent such as acetonitrile.

A suspensable small bead provides considerable advantages in diffusion time, amount of protein adsorbed, manipulation by filtration, sedimentation or attraction. Clumping of multiple beads by antibodies to different moieties on a protein may be minimized by agitating the beads to break such bonds or by using larger or porous beads with only the internal regions being coated.

The coating or the solid support is preferably hydrophobic as the hydrophilic portions of the protein will be presented for receptor binding as many antibodies typically bind to the hydrophilic portions of a protein molecule.

If the solid support denatures protein during the adsorption process, it is preferable to coat the solid support with an adsorption material that will not denature the protein and preferably maintain an aqueous environment. An example is a gel coating which immobilizes the protein such as the polyacrylamide gel pad used in Guschin et al, Analytical Biochemistry 250:203-211 (1997) or an amine or carboxyl reactive coating, particularly 3D-LINK by SurModics (Eden Prairie, Minn.) which is a hydrophilic amine reactive polymer topcoat on a silane base coat.

An alternative way to enhance binding of protein to the solid support is to use an avidin coated solid support. The protein sample is first biotinylated by known commercial procedures (e.g. Pierce). All of the proteins are then bound to the solid support through biotin-avidin bonds. Other protein derivatizing agents and receptors therefore may also be used such as dinitrophenol derivatives and anti-DNP antibody as the receptor.

Receptors may be immobilized on the solid support by first contacting and binding them to the ligands followed by the ligands binding to the solid support either non-specifically or through an affinity binding such as with biotinylated ligands and avidin coated solid support.

Other types of binding materials and methods can be used wherein one of the pair of molecules that is to be bound is modified to carry one member of a pair of molecules that forms a binding pair, and the other of the molecules that is to be bound is modified to carry the other member of the binding pair, as known in the art. Suitable binding pairs include, for example, avidin/biotin, as provided hereinabove, antibody/hapten (various modifications of antibody are possible so long as the antigen binding ability is maintained), antibody/F_{c} receptor (various modifications of antibody are possible so long as the F_{c} binding regions is maintained), receptor/ligand, receptor/hormone, lectin/carbohydrate and various chemicals, such as phenylboronic acid/salicylhydroxamic acid.

Separating the nucleic acids, particularly the sequence tags from the solid support may involve degradation of the ligands and/or receptors. This is acceptable, a number of nucleic acid extraction procedures are well known and commercial kits are available from multiple manufacturers including Qiagen. For example Sambrook et al, Molecular Cloning, 2^{nd} ed., Cold Spring Harbor Press, Cold Spring Harbor, N.Y. (1989).

The detection of and quantification of the nucleic acids containing sequence tags may be performed by a variety of techniques known per se for detecting and quantifying nucleic acid mixtures. The most common technique is with a microarray containing a large number of different oligonucleotides or nucleic acids where each one is located at a specific addressable location. Examples of such include the U.S. Patents cited above. The nucleic acids containing sequence tags of the present invention are contacted with a microarray under suitable conditions to allow specific hybridization to occur. From the particular locations and quantity of nucleic acids hybridizing to the microarray, one can deduce which ligands were present in the same and their concentration.

Other microarrays having cloned or amplified DNA deposited on a glass or other surface in an array may be used also. Frequently using cDNAs, a number of companies sell such synthesized microarrays. These microarrays may also be used. See Brown et al, U.S. Pat. No. 5,807,522.

For microarrays that are not a unitary solid phase, multiple different beads, each with a different label or having a different combination of labels may be used. For example, a bead having different shades of a chromagen or different proportions of different chromagens. Each bead or set of beads with the same identifying label(s) is to have an immobilized nucleic acid of a particular known sequence. Individual sets of beads may be identified in a mixture by spreading on a flat surface and scanning. The combination of the sequence tag label and the bead label(s) provides identification of the ligand of interest in the sample. The numerical ratio of beads having sequence tags hybridized thereto provides a quantitative measurement. Just as the sequence tag may be deduced from which cells contained hybridized sequence tags in a traditional microarray, with plural unique beads, the sequence may be deduced by determining which bead contains the sequence tag.

If so desired, the antibody display phage may be prescreened using the methods of U.S. Pat. No. 5,580,717 to preselect desired display antibodies. Also, the addition of the specific sequence tags may be added during such a process.

In another preferred embodiment of the present invention, the sequence tags are chosen to have at least part of the sequence of or complementary to the DNA or mRNA sequence encoding the protein being detected. Most preferable are sequence tags having a sequence complementary to the nucleic acid probes immobilized on a conventional gene array. Conventional gene arrays have immobilized nucleic acids complementary to many genes expected to produce a protein in a sample. Using a sequence tag complementary to these immobilized nucleic acids permits one to quantify proteins using the same software as is used to quantify mRNA in a sample. The sequence tags of the present invention need only provide a unique identifier and not be lengthy.

As an alternative method to detecting specific sequence tags hybridized to an immobilized nucleic acid of known sequence, one can detect specific sequence tags by whether or not specific amplification can be performed. In this situation, complementary primers to the specific sequence tags and a common primer to a common region of the phage are used in a PCR reaction. The absence of specific sequence tags makes the targets incapable of amplification. Such an amplification resistant multiplication system (ARMS) has previously been used to determine the presence of mutations in specific target nucleic acids based on which primer pairs is involved in amplification.

It should be noted that a one to one molecular correlation should be present between ligand molecule and sequence tag. Exceptions occur when the ligand has plural different or identical receptor binding sites or when the ligand is a polymer.

Microarray technology can employ a number of different detection systems. One of the simplest is by using labeled nucleic acids containing sequence tags. When hybridized, microarray cells having bound sequence tags will have the detectable label. Alternatively, either the target or the array's nucleic acids or oligonucleotides may prime the other for extension with polymerase and labeled NTP. Alternatively, the immobilized oligonucleotides or nucleic acids on the microarray may be labeled and cleaved and loose a label only when hybridized to the target. Numerous other microarray arrangements are known per se for detecting other nucleic acids and such arrangements may be used in the present invention as well.

Amplification of low concentration nucleic acids containing sequence tags is typically performed prior to hybridization on a microarray. However, low concentrations may be compensated for even after hybridization by using a signal enhancing system to amplify the signal. One such technique is by hybridizing additional labeled nucleic acids to a region of the nucleic acid containing sequence tags not already hybridized to the microarray. Another technique is to hybridize a circular nucleic acid to this region and add a strand displacing polymerase and labeled NTP. This results in a rolling circle amplification localized at a specific location. See Lizardi et al, Nature Genetics 19:225-232 (1998). A number of other techniques are known for quantifying nucleic acids such as FRET labeled hairpin probes (U.S. Pat. No. 5,925,517) and primers (U.S. Pat. No. 5,866,336).

To better quantify the proteins in the sample, the nucleic acids containing the sequence tags may be amplified to different levels or once amplified, many be diluted. Each sample may then be quantified as above. Since many nucleic acid detection techniques, particularly a microarray are less than ideally quantifiable, by using different concentration samples, one can better determine the quantity of each protein when they are in vastly different concentrations.

Critical to the functioning of the present invention is a reagent that contains a plurality of binding components each having a receptor that specifically binds to a ligand in association with a nucleic acid containing a unique sequence tag. The receptor and nucleic acid may be chemically linked such as a nucleic acid label conjugated to an antibody. More preferred is a physical attachment as in the situation of an antibody or other heterologous receptor expressing biological cell or microorganism. The reagent generally contains hundreds or thousands of different binding components, ideally corresponding to and specifically binding to at least every ligand in the sample being tested.

In the preferred embodiment, the reagent contains recombinant bacteriophage carrying antibody molecules on their surface, and incorporating DNA that includes an antibody-specific sequence tag. The surface antibodies are present as coat protein fusion products produced by well-known methods of phage display. Sampath et al, Gene 190(1): 5-10 (1997). The antibody-specific sequence tag is a short (e.g., 20 nucleotides) synthetic sequence uniquely associated with the antibody sequence (hence with its specificity) and introduced into the phage genome by recombinant methods. The sequence and perhaps nearby sequences are preferably flanked by restriction sites for easy excision and to know which primer set to use to amplify the sequence tag if desired. Such phages are bound to the solid support by interaction with target proteins previously attached thereto. Therefore, an amount of each sequence tag bound is related to the amount of its target protein in the sample. Unbound phages are removed from the support by washing steps, so that only the bound phages remain.

The nucleic acids indirectly bound to the solid support may be recovered by first striping all phage from the solid support with a pH change, such as an acidic buffer, or other denaturing conditions and then the nucleic acid recovered from the phage. Bacteriophages typically used for phage display are stable up to pH 11. One can easily elute antibody display phage from the bound antigen by a high pH buffer. Alternatively, the extraction of nucleic acids may be performed in-situ. This alternative is preferred when using small beads as the solid support as their removal provides an easy technique for removing at least some of the protein.

The separation of bound receptors from unbound receptors may be done by techniques other than being bound to a solid support ligand. For example, the ligand may be free while binding to the receptor followed by adding another reagent that will precipitate the ligand-receptor complex or free receptors to effect removal. Furthermore, filtration, electrophoresis or other techniques may separate the ligand-receptor complex from the unbound receptors.

The nucleic acid may be used directly, labeled and/or a fragment containing the sequence tag cleaved first. To cleave the sequence tag free of most of the remaining nucleic acid, restriction endonucleases are generally used; preferable cleaving at one or two unique sites somewhat adjacent to the sequence tag. To label the nucleic acid one may use end labeling with a label such as a direct fluorescent molecule or small molecule, which binds to a labeling compound such as digoxigenin. The end labeling may be by chemical addition of a fluorescent moiety or by adding a fluorescence labeled nucleotide with terminal deoxynucleotidyl transferase. See Sambrook et al, Molecular Cloning, 2^{nd} ed., Cold Spring Harbor Press, Cold Spring Harbor, N.Y. (1989). Other labeling techniques may also be used such as nick translation or with a labeled antibody to double stranded nucleic acids added later after hybridization to the nucleic acids in the microarray. The nucleic acid may also be used as a primer, which is extended in a system where labeled NTP and polymerase result in a labeled sequence tag. When the sequence tag is used as a primer, almost any template may be used with another primer to the template. More preferably, the template contains the complement to the sequence tag and a sequence of only one nucleotide before coding for the reverse sequence. This will permit significant amounts of label incorporation in a short sequence.

In the situation where the sample is expected to contain low concentrations of a particular ligand, one may amplify the sequence tag (and adjacent sequences) to obtain easily identifiable amounts of sequence tag. Primers to the sequence tag or adjacent sequences are preferred. The amplification process is a convenient step for simultaneously labeling the sequence tag(s) by standard protocols for labeling during amplification.

The nucleic acids containing the sequence tag(s) is then contacted with a detection system such as a microarray, blot or a series of unique solid phase particles. Oligonucleotide microarrays such as those manufactured by Affymetrix are preferred but any immobilized nucleic acid array may be used. The sequence tags are then allowed to specifically hybridize to the immobilized nucleic acids. If the sequence tag containing nucleic acids are not already labeled (or label removed) a detection labeling system is employed to label or unlabel the cell containing the sequence tag. This may be done by adding a labeled probe, amplifying nucleic acids in the cell, cleaving a label free from nucleic acids in the array cell or otherwise rendering cells containing immobilized sequence tags detectable or distinguishable from cells of the array not containing sequence tags. A sequence tag does not actually need to remain immobilized as long as it has performed its function of altering the labeling status of its corresponding cell of the array. For example, if the array contains end labeled oligonucleotides, the sequence tag hybridizes thereto forming a restriction site and an endonuclease is added to cleave the label free from the immobilized oligonucleotides. In such a situation, the sequence tag may be cleaved and washed free of the microarray and/or the remaining portion of the sequence tag may no longer anneal to the remaining immobilized oligonucleotide portion.

Since the nucleotide sequence tags are selectable before beginning the assay, one may use random sequences or predetermined sequences. Predetermined sequences are chosen to be complementary to the immobilized nucleic acids on the array. In such an arrangement, one may use “off the shelf” microarrays used for very different purposes with custom sequence tags or vice versa. Alternatively, microarrays with predefined optimized or random sequences are usable. As exemplified below, commercial p53 microarrays with thousands of different cells containing different oligonucleotides are used. The fact that these microarrays were commercially used to detect mutations and polymorphisms in the p53 gene is irrelevant as for the present invention; all that is needed is an array of many different nucleic acids of known sequences. Alternatively, commercial p53 mutation oligonucleotide microarrays may be used where the sequence tags correspond to the p53 mutations known from the Soussi database.

Very recently, after filing the priority document, Affymetrix, Inc. has begun selling GeneFlex Tag Array microarrays where the oligonucleotides correspond to unique sequence tags. These are 20 bases long and are selected from all possible 20 mers to have similar hybridization characteristics and minimal homology to sequences in the public databases. This microarray and other comparable ones are preferred embodiments of the present invention and may be used in the present method.

Once the array has the labeling altered at selected cells corresponding to the sequence tags, different labeling in different array cells is determined. This is done in a manner dependent on the nature of the label. For light emitting (fluorescent, chemiluminescent etc.) or light adsorbing (chromagen generation, precipitation or adhering in the cell) labels, optical scanning of the microarray may be employed. Confocal microscopic optical scanners are currently being used for scanning microarrays for conventional uses. Other detection systems may also be used such as those determining electrical properties. When the label alters an electrical property, such as resistaice, this is detectable from electrode probes and/or electrode containing microarray cells such as those in Okano et al, U.S. Pat. Nos. 5,434,049 and 5,607,646 and Thorp, U.S. Pat. No. 5,968,745. Radioactive and other labeled probes may also be used and the presence or absence of the label may be detected.

Important to the labeling and detection systems is the ability to determine quantity of label present to quantify the ligands present in the original sample. The detection system will depend on the specific label. Since the signal and its intensity is a measure of the number of sequence tags in the bound DNA sample and hence of the number of receptors bound, the number of ligand molecules in the original sample may be determined. Optical and electrical signals are readily quantifiable. Radioactive signals may also be quantifiable directly but preferably is determined optically by use of a standard scintillation cocktail. Enzyme labels may catalyze a large number of different reactions removing a substrate or producing a product that is readily detectable to produce a signal by any of the spectorphotometric, electrical or other techniques mentioned above. Even in situations where the sequence tag has been amplified, a quantitative measurement may be calculated.

While the receptors utilized in the examples are antibody molecules, one may equally use other specific binding receptors such as hormone receptors, certain cellular surface proteins (also called RECEPTORS in the scientific literature), an assortment of enzymes, signal transduction and binding proteins found in biological systems.

Likewise, ligands exemplified as proteins below may also be small organic molecules such as metabolic products in a biological cell. By simultaneously detecting many or all metabolites in a sample, one can determine the global effects of an effector on the cell. Effectors may be drugs, toxins, infectious agents, physiological stress, environmental changes, etc.

Conventionally, to determine the effect of a compound on a tissue, cell or biological system, the compound is added and a single or few products are measured. While such an approach is acceptable if one wishes to optimize production of a single product from the system (e.g. penicillin production from culture), this approach will not determine how a toxin affects the entire metabolism of a biological cell. The present invention permits one to determine such global effects on the cell by using a reagent containing receptors for many or all metabolites in a metabolic pathway. When the ligands being bound are small molecules involved in metabolic pathways, one may use a large number of enzymes and other interacting proteins to completely map the metabolic pathway to determine the effects of a drug or toxin on each step in the metabolic pathway.

The samples may be from environmental sources, different strains of life forms, manufactured mixtures, etc. Particularly preferred samples are those taken from a manufacturing process wherein the present invention is used for quality control. Representative manufacturing processes include chemical, pharmaceutical, food, feed, biologics and specialty chemicals.

As an alternative to amplifying a sequence tag after nucleic acids are separated, one may design the sequence tag region prior to beginning the assay. To detect proteins of low abundance relative to others, multiple tandem repeats of the sequence tag region can be incorporated into the phage genome separated by restriction enzyme sites. Thus, the receptor for a specific low abundance protein may contain, for example, 10 copies of its associated sequence tag per receptor. When the nucleic acids are freed from the receptors, an amplification factor of 10 will be produced after restriction enzyme cleavage compared to binding reagents with only one copy of the sequence tag per receptor.

Alternatively, low abundance proteins can be detected by altering the type and/or increasing the number of label moieties on the sequence tags containing nucleic acids. This may be done by selective amplification of nucleic acids having only certain sequence tags, using a different (or additional) labeling technique for certain sequence tag containing nucleic acids, or by adding an additional label at a later point in the process. For example, a template labeled NTPs and polymerase are added to label all nucleic acids containing sequence tags. Additionally or preferably subsequently, a second set of templates which is primed by only nucleic acids containing certain sequence tags (those corresponding to low abundance proteins) may be added with another or differently labeled NTP(s) for further labeling. Alternatively, one can add a labeled oligonucleotide that will hybridize to the sequence tags corresponding to low abundance ligands after the nucleic acid is hybridized to the microarray to provide additional label signals to that cell.

While it is very useful to know the quantities of various ligands in a sample, in some situations, one may find it useful to compare the sample to a standard or to measure differences in concentrations of various ligands from another sample. For example, disease specific makers may be deduced by determining which proteins are in higher or lower concentrations in a sample from diseased tissue as compared to normal tissue. The differential may be determined by using the present invention to determine the quantities of sequence tags in a normal and a diseased sample. The results from each experiment are compared to generate the differential results.

The present invention may also determine the differential results directly without actually determining the concentrations of any ligand in either sample. This is done by using a single stranded nucleic acid virus as the receptor display system. Two sets of sequence tags are used, one for the normal sample and one from the diseased sample. The only difference in the reagents is that the sequence tags in the reagent for the diseased sample are complementary to the sequence tags in the reagent for the normal sample. Both assays are run separately and may be simultaneously in separate containers. However, the final steps of contacting microarray are omitted. Instead, the two pools of sequence tags are mixed together under hybridizing conditions. Double stranded nucleic acids are removed or inactivated so that only differential single stranded nucleic acids remain. The differential nucleic acids are then contacted with the microarray and the process continued to yield a differential result.

Common concentrations of each ligand in the two samples are effectively nullified by being removed by a number of conventional techniques such as a hydroxyapatite column, antibody to double stranded nucleic acids, DS-DNase (especially endonucleases) or crosslinking of double strands with UV or chemical methods. If only one of over or under concentration is to be measured, one may perform a subtraction procedure by biotinylating one pool (with a lesser abundance) of sequence tags before mixing and then after hybridization, contacting it to avidin immobilized on a solid phase to separate and remove the double stranded nucleic acids.

Particularly preferred is to label one pool of sequence tags with a different label from the other pool of sequence tags. For example, if one pool is labeled with fluorescein and the other is labeled with rhodamine, the differential results can easily be calculated when scanning the microarray for each fluorescent signal wavelength.

Determination of differential concentrations between two samples is helpful in identifying disease specific markers, plant and animal breeding, and a large number of analytical and diagnostic determinations.

While antibody display bacteriophage are well known and used for a variety of other purposes, they are not the only suitable nucleic acid labeled receptor that may be used. Other microorganisms or even cells may be used such as E. coli containing antibody or other receptor genes cloned in a plasmid, cosmid, BAC or integrated into the genome, yeast particles containing a receptor or antibody gene a wide assortment of viruses and subcellular particles. See Protein Engineering 12(7): 613-21 (1999). Generally, smaller particles are preferentially used, as attachment to the ligand must immobilize a particle. In any situation, the antibody, or other receptor, should be produced in such a fashion that it will be effective to bind the ligand.

Theoretically, one may even use antibody displaying hybridomas in lieu of antibody display phage. However, incorporating a known quantity of sequence tag into such an antibody-producing cell is difficult as they are tumor cells and genetically unstable with aneuploidy or independent replication of plasmids generating a variable number of sequence tags per cell. Cells of comparable size have been removed from suspensions by antibody/antigen interactions on a solid support many years ago by Edelman et al.

As an alternative to using antibody display phage, one may use a receptor, such as an antibody molecule, conjugated to a nucleic acid containing the sequence tag. A cleavable linker between the receptor and the nucleic acid is preferred. The method proceeds as above with minor modifications to the step of releasing the sequence tag from the immobilized receptor. In this situation, the receptor and nucleic acid containing sequence tags will be known beforehand and individually synthesized. The assay is initially performed in the same manner as any other conventional immunoassay using a labeled reagent. Of course, the analytes are plural and the detection system is quite different.

When antibody display phages are produced with the same antibody binding domain and different sequence tags, the phage may be reinfected and a single plaque used as the phage.

Since the specific binding of ligand to receptor is structure specific, two or more small differences in the ligand may be separately detectable. For example, proteins in the same sample may contains the same general protein with different post-translational modification such as differential splicing, glycosylation, phosphorylation, cleavage and agglomeration into a quaternary structure or protein complex. Each variant may be separately detectable and quantifiable by binding to different receptors. Likewise for compound congeners and antibodies differing only in the variable portion of the molecule.

Another embodiment of the present invention is to use a sequence tag labeled nucleic acid probe or primer to detect and/or quantify the number of copies of a target nucleic acid in a sample. This may be viewed as a sequence tag labeled probe or primer used to detect and/or quantify a complementary target nucleic acid. In this arrangement, the sample contains a mixture of multiple target nucleic acids. A representative example is plural mRNA from a biological sample. The nucleic acid sequence tag labeled receptor used as a reagent has a complementary nucleic acid sequence to each of the target nucleic acids being measured. The sequence tag and receptor may be chemically bound or otherwise physically attached. By first immobilizing the target nucleic acids, the amount of each reagent containing a sequence tag bound is proportional to the amount of each target. The sequence tag is then separated and detected as in the general method above. The preferred use for this embodiment is to simultaneously measure the quantity of many mRNA molecules in a biological sample in order to determine the state of a cell's or tissue's metabolism. This is an alternative to the known technique of measuring the quantity of each mRNA by directly hybridizing it to the microarray.

While hybridization and Watson-Crick binding are discussed, it is contemplated that one can use triple strand or Hoogstein binding in lieu of complementarity. If binding has sufficient specificity, it may be used in the present invention.

The following examples are included for purposes of illustrating certain aspects of the invention and should not be construed as limiting.

### EXAMPLE 1

### Synthesis of Antibody Phage Display Libraries Having Unique Sequence Tags

Human serum is used as the immunogen in the antibody display phage procedure of Winter et al, Annual Review of Immunology 12: 433-55 (1994) modified as follows. The mRNA is separated and cloned into M13 phage according to the techniques of Sampath et al, Gene 190(1): 5-10 (1997). The mixed DNA containing antibody domains are blunt ligated, to a mixture of 18 base sequence tags at a restriction endonuclease site in the middle of the beta-galactosidase gene. Each sequence tag has a sequence of an 18 base sequence of the p53 gene from nucleotide number 1x to 1x+18 where x is 5 or a multiple of 5. The sequence of the p53 gene is well known and provided with the off-the-shelf p53 GENECHIP. The ligation is random, yielding phage containing vectors having a large number of phage with a large number of different sequence tags. Selection of individual blue colonies from transformed bacteria is used followed by formation of the library with helper phage.

The AFFYMETRIX p53 GENECHIP having oligonucleotides to the entire sequence of p53 is used. The sequence tags of 18 mers are complementary to the immobilized 18 mers of the microarray. The sequence overlap is no more than 13 bases except in exact matches.

### EXAMPLE 2

### Simultaneously Quantitative Detection of Numerous Serum Proteins

Human serum samples are taken from two human volunteers, one normal healthy male and another male having active hepatitis infection. Each sample is diluted 100 fold and allowed to adsorb on the inner surface of a plastic tube for one hour at room temperature. The sample is decanted, washed twice with saline and fish skin gelatin blocking agent is added to the tube and incubated for one hour at room temperature. The solution is decanted and washed twice with saline.

The antibody display phage library of Example 1 is diluted added to the tube and incubated for one hour at room temperature. The concentration of the phage is adjusted to be in vast molar excess. The solution was decanted and washed four times with TRIS buffered saline. A 0.1% pronase solution is added and incubated overnight in a 37° C. water bath. DNA is extracted from the resulting solution using a QUAGEN Miniprep™ extraction procedure. The DNA is cleaved with the same restriction endonuclease as in Example 1 and electrophoresed in a polyacrylamide gel. The low molecular weight band is removed, eluted and end labeled with fluorescein labeled DATP via terminal transferase (TdT). The other sample's nucleic acids are labeled with rhodamine labeled dATP via TDT. These labeled nucleic acids are pooled and hybridized to the p53 GENECHIP and scanned according to the instructions. The microarrays are scanned for fluorescence for one label at a time and the results reported numerically for each cell of the microarray. In addition, the computer is instructed to subtract one fluorescence signal from the other fluorescence signal to obtain differential values for each protein. By measuring the concentration of a typical known protein in human serum, a pattern of the relative concentrations of each protein is developed.

### EXAMPLE 3

### Diagnostic Testing of an Unknown

Serum samples from subjects with active hepatitis and healthy subjects are treated as in Examples 1 and 2 above. The results are compared to the patterns demonstrated by the normal and hepatitis subject of Example 2 and scored appropriately to determine which serum sample is positive. Even though the samples are from subjects with different forms of hepatitis, certain protein concentrations changes common to hepatitis are observable.

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto.

All patents and references cited herein are explicitly incorporated by reference in their entirety.

1. A microarray having immobilized thereon a plurality of oligonucleotides complementary to sequence tags.

2. The microarray of claim 1 wherein the sequence tags have a random sequence.

3. A recombinant microorganism capable of expressing a specific receptor on its surface and containing a unique nucleic acid sequence tag.

4. A plurality of different recombinant microorganisms according to claim 3 wherein each different microorganism contains a different specific receptor and a different nucleic acid sequence tag.

5. The recombinant microorganism of claim 3 wherein the sequence tag is part of a nucleic acid containing at least part of an antibody gene.

6. The recombinant microorganism of claim 3 wherein the sequence tag is part of a nucleic acid containing at least part of a microorganism or cellular gene.

7. A nucleic acid labeled receptor comprising;
a specific binding receptor, and
a nucleic acid containing at least 13 nucleotides,
wherein the nucleic acid is physically or chemically bound to the specific binding receptor.

8. A plurality of nucleic acid labeled receptors according to claim 7 wherein each receptor specifically binds to a different ligand and is labeled with a nucleic acid having a different sequence.

9. The nucleic acid labeled receptor of claim 7 wherein the sequence tag is part of a nucleic acid containing at least part of an antibody gene.

10. A microarray comprising;
a solid phase containing a plurality of cells in a definable location,
a plurality of nucleic acids immobilized on the solid phase, wherein each cell of the solid phase contains all of the nucleic acids of a particular sequence, and
a nucleic acid sequence tag specifically hybridized to the nucleic acid.

11. The microarray of claim 10 wherein a plurality of nucleic acid sequence tags, each with a different nucleotide sequence, are hybridized to a plurality of different cells wherein all nucleic acid sequence tags of the same sequence are hybridized in the same cell of the solid phase.

12. The microarray of claim 10 wherein a plurality of discrete solid phase particles constitute the solid phase and wherein each of said particles constitute the cell.

13. The microarray of claim 10 wherein the sequence tag is part of a nucleic acid containing at least part of an antibody gene.

14. The microarray of claim 10 wherein the oligonucleotide sequence tag is part of a nucleic acid containing at least part of a microorganism or cellular gene.

15. A microarray comprising;
a solid phase containing a plurality of cells in a definable location,
a plurality of nucleic acids immobilized on the solid phase, wherein each cell of the solid phase contains all of the nucleic acids of a particular sequence and wherein a nucleic acid sequence for each of the nucleic acids is complementary to predefined sequence tags, each with a different nucleotide sequence.

16. The microarray of claim 15 wherein a plurality of discrete solid phase particles constitute the solid phase and wherein each of said particles constitute the cell.

17. The microarray of claim 15 wherein the sequence tag is part of a nucleic acid containing at least part of an antibody gene.

18. The microarray of claim 15 wherein the oligonucleotide sequence tag is part of a nucleic acid containing at least part of a microorganism or cellular gene.

19. A method of determining the presence of a ligand in a sample of mixture of different ligands comprising;
contacting at least one recombinant microorganism of claim 3 or the receptor of claim 7 under conditions suitable for binding of ligand to receptor,
separating bound receptors from unbound receptors,
detecting the presence of at least one sequence tag.

20. The method of claim 19 further comprising quantitatively determining the amount of the ligand in the mixture by determining the quantity of sequence tag from bound receptors.

21. The method of claim 18 further comprising simultaneously detecting the presence of plural different ligands in the sample by simultaneously detecting the presence of corresponding different sequence tags.

22. The method of claim 21 wherein the concentration of one ligand being detected is at a concentration at least ten fold greater than another ligand being detected in the sample.

23. The method of claim 22 further comprising quantitatively determining the amount of both ligands in the mixture by determining the quantity of sequence tags from bound receptors

24. The method of claim 19 further comprising labeling the nucleic acid containing the sequence tag.

25. The method of claim 19 wherein the presence of the nucleic acid containing sequence tag is detected by specific hybridization to a plurality of complementary nucleic acids which are physically separated or separable from each other such that one can determine which are hybridized.

26. The method of claim 25 in which said complementary nucleic acids are located in an array on a solid phase.

27. The method of claim 19 further comprising amplifying the number of molecules of nucleic acid containing the sequence tag.

28. The method of claim 19 wherein the ligands are proteins and the receptors are proteins expressed from a gene derived from an antibody.

29. The method of claim 19 wherein the receptor is on the surface of a virus.

30. The method of claim 27 wherein the nucleic acid containing the sequence tag is amplified by annealing to a primer and extending the primer.

31. The method of claim 19 further comprising the step of initially adding a known quantity of a control ligand to the sample wherein the concentrations of all other ligands in the sample may be determined relative to the control ligand.

32. A solid support having a plurality of ligands immobilized thereon and a plurality of receptors of claim 7 bound to the ligands.

33. A solid support having bound thereto a plurality of different recombinant microorganisms capable of expressing a specific receptor on its surface wherein the recombinant microorganism contains a heterologous gene encoding the receptor.

34. A solid support of claim 33 wherein the solid support is bound to a ligand and the ligand is bound to the receptor on the recombinant microorganism.

35. A method for fractionating a mixture of recombinant microorganisms, each capable of expressing a different specific receptor on a surface thereof comprising;
contacting the mixture with a solid support and allowing at least part of the mixture to become bound thereto,
removing unbound recombinant microorganisms.

36. The method of claim 35 further comprising eluting bound recombinant microorganisms from the solid support.

37. The method of claim 35 wherein the recombinant microorganisms are bound by the receptor to ligands immobilized on the solid support.

38. The method of claim 37 further comprising initially immobilizing ligands on the solid support.

39. The method of claim 37 further comprising binding the receptor to the ligands followed by immobilizing the ligands on the solid support.
